(19) United States
(12) Patent Application Publication
(10) Pub. No.: US 2017/0242006 A1
(43) Pub. Date: Aug. 24, 2017

(54) PREDICTION OF THE SUSCEPTIBILITY OF AN AT RISK PATIENT FOR DEVELOPING OR REDEVELOPING CLOSTRIDIUM DIFFICILE INFECTION

(71) Applicant: BIOMERIEUX, Marcy L'Etoile (FR)

(72) Inventors: Agnès FOUSSADIER, Lyon (FR); Mark MILLER, Lyon (FR)

(73) Assignee: BIOMERIEUX, Marcy L'Etoile (FR)

(21) Appl. No.: 15/519,724

(22) PCT Filed: Oct. 16, 2015
(86) PCT No.: PCT/FR2015/052778

§ 371 Date: Apr. 17, 2017

(30) Foreign Application Priority Data
Oct. 17, 2014 (FR) 1459996

(51) Int. Cl. G01N 33/569

(52) U.S. Cl. CPC G01N 33/56911; G01N 2333/33; G01N 2800/54; G01N 2469/20

(57) ABSTRACT

A method for prediction of the susceptibility of an at risk patient to developing or redeveloping an infection with Clostridium difficile, having the determination by immunoassay, in a stool sample from said patient, of the level of antibody IgA anti-toxin B of Clostridium difficile, and comparing this level with a reference value S determined beforehand using two populations of patients exposed to the bacterium, one population not having developed or redeveloped such an infection and the other population having developed or redeveloped such an infection, —a level lower than said reference value S signifying that the patient is a patient with a heightened risk of developing or redeveloping a Clostridium difficile infection, and —a level higher than said reference value S signifying that the patient is not a patient with a heightened risk of developing or redeveloping a Clostridium difficile infection.

The present invention relates to the field of infections by the bacterium Clostridium difficile. In particular, the invention relates to prediction of the susceptibility for developing or redeveloping infections due to the bacterium Clostridium difficile.

The bacterium Clostridium difficile is a toxinogenic, sporulating, anaerobic bacterium, of the Gram+ bacillus type. It is an enteropathogen that causes post-antibiotic pseudomembranous colitis (PMC) or diarrhea, and is mainly involved in nosocomial diarrhea in adults. It is formidable on account of its very high potential for contagion. Although about 5% of the population are asymptomatic carriers of the bacterium, its pathological manifestations are closely linked to a stay in hospital.

This bacterium develops in an intestinal flora weakened by antibiotic therapy and may secrete two toxins, toxin A and toxin B. Only the strains producing toxins are pathogenic. Toxin A, an enterotoxin, causes alteration of the permeability of the intestinal epithelium. Toxin B, a cytotoxin, attacks the cells of the epithelium directly. The combined effect of the two toxins is reduction of the intestinal transit time and of intestinal absorption, resulting in diarrhea.

Some years ago, the bacterium Clostridium difficile was not regarded as a clinical nuisance. Since 2003, the emergence and rapid spread of hypervirulent strains such as the strain C. difficile Ribotype 027 in the United States, in Canada, and then in Europe, led to public health vigilance for this bacterium (Kuijper E. J. et al., 2006). Today it is the primary cause of nosocomial infectious diarrhea, responsible for 15% to 25% of cases of pseudomembranous colitis (Bartlett J. G., 2002). It causes increasingly severe cases and increased mortality in general hospitals. Every day, 7 to 12 patients out of 10000 are affected in the United States (Freeman J. et al, 2010) and up to 5 patients out of 10000 in Europe (Bauer M. P. et al., 2011),

Diagnosis of infection with Clostridium difficile in stool samples is known. It consists of several steps. The first step, which is not obligatory, consists of investigating for one of the detectable proteins representative of the presence of this bacterium, namely glutamate dehydrogenase (GDH). Other detectable proteins are the toxins that are secreted when the bacteria are toxinogenic. Detection or quantification of GDH may be carried out by immunoassay, for example by ELISA assay. This technique gives greater diagnostic sensitivity than detection or quantification by immunoassay of the toxins. If the test for GDH is positive, investigation for the toxins is then recommended, as this technique offers greater specificity.

This investigation for toxins in a stool sample constitutes the second step, or the first if investigation for GDH is not performed. It may be carried out by ELISA assay or by an assay for cellular cytotoxicity, the latter constituting the “gold standard”. It is also possible to use PCR, which is more sensitive. The main drawback of this technique is that it only detects the presence of the genes of toxins A and/or B, without indicating whether the toxin is actually produced, which may be detrimental to specificity. In fact, the production of toxins may be inactivated, and in this case the strain is not pathogenic.

Finally, if investigation for the toxin in the stool sample is negative, a bacterial culture is carried out in a suitable CCFA (Cycloserine Cefoxitin Fructose Agar) medium, which constitutes the third step. If the culture is positive, investigation for the toxins is carried out on the colonies, for example by ELISA assay or by assay for cellular cytotoxicity.

After it is confirmed that the patient has a Clostridium difficile infection, he receives treatment. This treatment involves antibiotics such as metronidazole (MTZ) or vancomycin (VA). However, treatment failures are frequent and the reinfection rates reach 20% in the initial episodes and from 40% to 60% in successive infections (C. P. Kelly and J. T. Lamont, 2008 and D. W. Eyre et al., 2012). Reinfection normally occurs within 28 days after the end of treatment of the primary infection but the delay may be up to 6-8 weeks.

In 1994, Warny M., et al. (Wanly M. et al., 1994) studied the human immune response to toxin A of Clostridium difficile in 21 non-immunodepressed patients who had a single episode of infection with C. difficile and in 4 non-immunodepressed patients with (multiple) reinfection. In this study, the authors observed that the titers of anti-toxin A serum IgG antibodies and of anti-toxin A fecal IgA antibodies were significantly higher in the patients who had a single episode of Clostridium difficile infection than in the patients with (multiple) reinfection. This latter circumstance, obtained for a very small number of patients, was not investigated further. Another team (Johnson S. et al., 1992) even obtained opposite results, in the sense that they found that the patients with reinfection had a high titer of anti-toxin A serum antibodies.

A scoring system was elaborated for predicting which patients are susceptible for developing Clostridium difficile infection. This scoring system is based on several criteria, such as the patient's age, hospitalization, administration of antibiotics, etc. (Garey K. W. et al., 2008). However, the results from scoring do not always agree, so that the clinician still does not have access to a simple tool such as an in-vitro diagnostic test.

No team has yet succeeded in proposing an in-vitro assay that would make it possible to predict, among patients at risk, those susceptible for developing or redeveloping Clostridium difficile infection.

There is therefore an urgent need, unmet for many years, to find good tools so as to be able to predict, in patients at risk, those with a risk of developing or redeveloping Clostridium difficile infection.

The applicant discovered against all expectation that it was possible to use the type IgA fecal antibodies, directed against toxin B of Clostridium difficile, as a marker for identifying said at-risk patients.

Thus, the invention relates to a method for predicting the susceptibility of an at-risk patient for developing or redeveloping Clostridium difficile infection, comprising or consisting of determining by immunoassay, in a stool sample from said patient, the level of IgA antibodies against toxin B of Clostridium difficile, and of comparing this level with a reference value S previously determined with two populations of patients exposed to the bacterium, one that has not developed or redeveloped such an infection and the other that has developed or redeveloped such an infection,
a level below said reference value S signifying that the patient is a patient with increased risk of developing or redeveloping Clostridium difficile infection, and
a level above said reference value S signifying that the patient is not a patient with increased risk of developing or redeveloping Clostridium difficile infection.

It also relates to a method for determining, by immunoassay, the level of at least one IgA antibody directed against a protein that is unaffected in the presence of acid, preferably directed against toxin B of Clostridium difficile, in a patient's biological sample that may contain said at least one IgA antibody. The method comprises or consists of bringing one or more binding partners to said at least one IgA antibody, used for performing the immunoassay, into contact with an acidic reaction mixture to comprising said biological sample pretreated with an acidic sample treatment buffer, without neutralization before it is used in the immunoassay.

The invention finally relates to a kit for determining, by immunoassay, the level of at least one IgA antibody directed against a protein that is unaffected in the presence of acid, notably directed against toxin B of Clostridium difficile, in a patient's biological sample that may contain said at least one IgA, notably a stool sample, comprising (i) one or more binding partners to said at least one IgA antibody for performing the immunoassay, and (ii) an acidic sample treatment buffer, it being understood that said kit does not contain any neutralizing solution.

The applicant therefore showed, against all expectation, that it was possible to predict the susceptibility of an at-risk patient for developing or redeveloping Clostridium difficile infection, by determining, by immunoassay, in a stool sample from said patient, the level of IgA antibodies against toxin B of Clostridium difficile.

“Developing Clostridium difficile infection” means that the patient develops the infection for the first time, regardless of the strain of Clostridium difficile.

“Redeveloping Clostridium difficile infection” means that the patient has a recurrence, i.e. a relapse (infection due to the same strain of Clostridium difficile) or a reinfection (infection due to a strain of Clostridium difficile different from the strain causing the primary infection).

“At-risk patient” means a vulnerable person. This patient may be vulnerable owing to his condition, for example because:

he is immunodepressed or may become immunodepressed following future drug treatment or on occurrence of a new disease;

he has just developed an infection, regardless of the microorganism, except Clostridium difficile; in this context he may have received antibiotic treatment;

he has just developed a Clostridium difficile infection; in this context he may have received antibiotic treatment. In this case, this patient could redevelop Clostridium difficile infection.

He may also be vulnerable owing to his age, because he is a premature baby, an infant or a person over 65 years of age.

He may also be vulnerable owing to his environment, because he is, or will be, in a place where Clostridium difficile bacteria may be present, such as in a hospital or in a care center.

Of course, a person may have only one of these risks of vulnerability or may accumulate them.

As nonlimiting examples of patients at risk, we may mention persons who have just had a Clostridium difficile infection, who are still in hospital. In this case, the method of the invention may be carried out at the end of the treatment (after about 7-10 days of treatment and up to 28 days after the end of the treatment). According to one embodiment, the patient at risk is a patient diagnosed as having had a Clostridium difficile infection, and the risk consists of redeveloping a new Clostridium difficile infection.

We may also mention persons known or not known to have had a Clostridium difficile infection, and arriving in the hospital, these persons having a risk of being on antibiotic treatment and/or on immunosuppressants. According to another embodiment, the patient at risk is a patient arriving in hospital and possibly receiving a drug leading to a decrease of his immune defenses.

We may also mention persons arriving in hospital who are aged over 65 years, or are immunodepressed, and with a risk of being on treatment with antibiotics and/or immunosuppressants, regardless of these persons' status for Clostridium difficile infection, i.e. never having had a Clostridium difficile infection, being a carrier of Clostridium difficile or having already had a Clostridium difficile infection.

Depending on the result of the method of the invention and the stratification of the patient, if he is considered to be a patient with increased risk of developing or redeveloping Clostridium difficile infection, the clinician will be able to decide on one or more of the following actions: administration of moderate antibiotic therapy, isolation of the patient to avoid any contamination, postponement of the immunosuppressant treatment, administration of preventive immunotherapy, vaccination.

The method for predicting the susceptibility of an at-risk patient for developing or redeveloping Clostridium difficile infection employs an immunoassay. An immunoassay is a test that is generally known by a person skilled in the art. Briefly, it consists of determining the level of analyte, in the present case IgA antibodies against toxin B of Clostridium difficile, using at least one binding partner to this analyte.

Of course, the prefix “immuno” in the term “immunoassay”, for example, is not to be regarded in the present application as strictly indicating that the binding partner is necessarily a partner of immunologic origin, such as an antibody or an antibody fragment. In fact, as is well known by a person skilled in the art, this term is used more widely for also denoting assays and methods in which the binding partner is not a partner of immunologic origin/nature but consists, for example, of a receptor of the analyte that we wish to detect and/or quantify. The essential condition is that the binding partner concerned should be capable of binding to the required analyte, in the present case of the nature of an antibody, preferably specifically. Thus, it is known to talk of the ELISA assay for assays that use binding partners that are not immunologic in the strict sense, more commonly called “ligand binding assay” in English, which could be translated into French as “essai utilisant la liaison à un ligand”, whereas the term “immuno” is included in the full title corresponding to the acronym ELISA. For the sake of clarity and uniformity, the term “immuno” is used in the present application to denote any biological analysis using at least one binding partner suitable for binding to the required analyte and for detecting and/or quantifying the latter, preferably specifically, even when said binding partner is not of an immunologic nature or origin in the strict sense.

“Binding partner to IgA antibodies directed against toxin B of Clostridium difficile”, also called IgA antibodies against toxin B of Clostridium difficile, means any molecule capable of binding to said IgAs. As examples of binding partners to the anti-toxin B IgAs, we may mention native or recombinant toxin B, toxin B fragments, anti-IgA antibodies or any other molecule that is known to interact with the anti-toxin B IgAs.

Toxin B of natural origin, also called native, may be obtained after culture of the bacterium Clostridium difficile and purification of the protein from the bacterial lysate. Recombinant toxin B may be obtained by genetic engineering, by techniques familiar to a person skilled in the art. This is described for example by Anderson B M et al., 1993. Native toxin B may be obtained from companies such as The Native Antigen Company (Upper Heyford, United Kingdom).

The binding partners of the antibody type are for example either polyclonal antibodies, or monoclonal antibodies, production of which is widely known by persons skilled in the art.

As examples of antibody fragments, we may mention the fragments Fab, Fab′, F(ab′)2 as well as scFv (single-chain variable fragment), dsFv (double-stranded variable fragment). These functional fragments may notably be obtained by genetic engineering.

Immunoassay consisting of determining the level of anti-toxin B IgA is a semiquantitative or quantitative assay widely known by a person skilled in the art, preferably employing two binding partners to the IgAs. One of the two partners may be coupled to a marker to form a conjugate or a tracer. The other binding partner may be captured on a solid support. We then speak of capture partner for the latter and detection partner for the former. Preferably, the capture partner is toxin B of Clostridium difficile and the detection partner is an anti-human IgA antibody.

The measured signal emitted in the immunoassay is then proportional to the amount of anti-toxin B IgA in the biological sample.

Marker means, notably, any molecule containing a group that reacts with a group of the binding partner, directly without chemical modification, or after chemical modification to include such a group, said molecule being capable of directly or indirectly generating a detectable signal. A nonexhaustive list of these direct detection markers consists of:
a enzymes that produce a signal that is detectable for example by colorimetry, fluorescence, luminescence, such as horseradish peroxidase, alkaline phosphatase, β-galactosidase, glucose-6-phosphate dehydrogenase,
chromophores such as fluorescent compounds, luminescent compounds, dyes,
radioactive molecules such as ³²P, ³⁵S or ¹²⁵I,
fluorescent molecules such as Alexa dyes or phycocyanins, and
electrochemiluminescent salts such as organometallic derivatives based on acridinium or ruthenium.

Indirect detection systems may also be used, for example ligands capable of reacting with an antiligand. The ligand then corresponds to the marker, to constitute the conjugate with the binding partner.

Ligand/antiligand pairs are familiar to a person skilled in the art, which is the case for example for the following pairs: biotin/streptavidin, hapten/antibody, antigen/antibody, peptide/antibody, sugar/lectin, polynucleotide/polynucleotide complement.

The antiligand may then be detectable directly by the direct detection markers described above or may itself be detectable by another ligand/antiligand pair, and so on.

These indirect detection systems may lead, in certain conditions, to amplification of the signal. This technique for signal amplification is familiar to a person skilled in the art, and reference may be made to the applicant's earlier patent applications FR 2781802 or WO 95/08000.

Depending on the type of labeling used, a person skilled in the art will add reagents for visualization of the labeling or emission of a signal that is detectable by any suitable type of measuring equipment, for example a spectrophotometer, a spectrofluorometer, a densitometer or a high definition camera.

The immunoassay may also comprise other steps known by a person skilled in the art, such as washing steps and incubation steps.

The immunoassay may be a one-step or a two-step assay, as is widely known by a person skilled in the art. Briefly, a one-step immunoassay comprises bringing the test sample into contact with the two binding partners simultaneously, whereas a two-step immunoassay comprises on the one hand bringing the test sample into contact with the first binding partner, then the analyte-first binding partner complex thus formed is brought into contact with the second binding partner.

The reference value S used in the method according to the invention is a value obtained beforehand with two populations of patients exposed to the bacterium Clostridium difficile, for example in a hospital environment, one not having developed or redeveloped such an infection and the other having developed or redeveloped such an infection. Said determination is widely known by a person skilled in the art. It consists notably of carrying out an immunoassay identical to that employed in the method of the invention, in stool samples from these two populations, and of determining the assay (signal) value for discriminating between these two populations.

When the reference value S is a quantity, titer or concentration of antibodies, the immunoassay employed is quantitative and the result from the method gives the quantity, titer or concentration of IgA antibodies. It is then necessary to correlate the signal obtained with the quantity, titer or concentration in the biological sample using a mathematical model previously established from a standard range. This standard range will be obtained beforehand in a known manner. Briefly, obtaining a standard range consists of measuring the signal generated by known, increasing amounts or concentrations of the anti-toxin B IgA antibodies, plotting the curve giving the signal as a function of the quantity, titer or concentration and finding a mathematical model that represents this relation as faithfully as possible. The mathematical model will be used for determining, by extrapolation, the quantities, titers or concentrations of unknown anti-toxin B IgA antibodies, contained in the biological sample to be tested.

The biological samples that possibly contain the IgAs against toxin B of Clostridium difficile useful in the sense of the invention are patients' stools samples. They may be the stool itself or a rectal enema. These samples may be used as they are in the method of the invention or may have undergone a pretreatment, the latter embodiment being preferred.

Stool samples are a matrix that is difficult to treat. IgAs may be involved in complexes consisting of mucus, proteins, toxins, particles, etc. According to one embodiment, the stool samples employed in the immunoassay were treated beforehand with an acidic sample treatment buffer. Acidic sample treatment buffer means a buffer having a pH between 1.5 and 3, preferably between 2 and 3, a pH of 2.5 being preferred.

As examples of acidic sample treatment buffers suitable for the purposes of the invention, we may mention hydrochloric acid-glycine buffer (pH 2.2-3), phosphate-citrate buffer (pH 2.2-3), hydrochloric acid-potassium chloride buffer (pH 2-2.2) and citric acid-sodium citrate buffer (pH 3).

The duration of pretreatment of the stool samples must be reasonable, a duration of at most 30 min being suitable. According to one embodiment, the duration of pretreatment of the stool or rectal enema samples is at most 30 min, preferably at most 20 min, 15 min, 10 min, 5 min, 3 min, 2 min, 1 min, 45 s, 30 s, 15 s or 10 s.

The biological sample thus treated then undergoes separation, notably by filtration or sedimentation, for example centrifugation, the filtrate or the supernatant containing the IgA of interest then being recovered, to be used in the immunoassay of the method of the invention.

Usually, when a biological sample undergoes an acid pretreatment, the reaction mixture thus obtained is then neutralized to raise the pH again before it is used in an immunoassay, to avoid alteration of the binding partners used for the immunoassay, notably the capture partners. Against all expectations, such a neutralizing step is not necessary in the method of the invention, so that, according to one embodiment, the sample pretreatment step does not include a step of neutralizing the reaction mixture before it is used in the immunoassay.

“Reaction mixture neutralizing step” means addition of a neutralizing solution, basic for an acidic solution to be neutralized, allowing the pH to be raised to around 7 so as not to alter the binding partners employed in the immunoassay. In the context of the invention, this step is therefore unnecessary.

It should be noted that all the other buffers used conventionally in an immunoassay, such as washing buffers or reaction buffers, are not neutralizing solutions, since their aim is not to bring the pH of the acidic reaction mixture back up to around 7.

Other markers may be used for aiding prediction of the susceptibility of an at-risk patient for developing or redeveloping Clostridium difficile infection. Such markers are for example IgA antibodies against toxin A of Clostridium difficile.

Determination of the level of IgA antibodies against toxin A of Clostridium difficile may be carried out similarly to that described for determining the level of anti-toxin B antibodies: in a stool sample, if necessary pretreated with an acidic sample treatment buffer, by performing an immunoassay.

As we have just seen, an immunoassay consisting of determining a level of IgA in a biological sample may be carried out in an acidic reaction mixture, against all expectations. Thus, the invention further relates to a method for determining, by immunoassay, the level of at least one IgA antibody in a patient's biological sample that may contain said at least one IgA antibody, comprising bringing one or more binding partners to said at least one IgA antibody, used for performing the immunoassay, into contact with an acidic reaction mixture comprising said biological sample pretreated with an acidic sample treatment buffer, without neutralization before it is used in the immunoassay.

In other words, the method for determining, by immunoassay, the level of at least one IgA antibody in a patient's biological sample that may contain said at least one IgA antibody comprises or consists of:
providing a patient's biological sample pretreated with an acidic sample treatment buffer,
providing an immunoassay kit comprising one or more binding partners to said at least one IgA antibody,
bringing said pretreated sample into contact with the binding partner or partners in said kit, it being understood that said pretreated sample does not undergo neutralization before it is brought into contact with the binding partner or partners, and
determining the level of IgA antibodies.

The IgA antibodies suitable in the sense of the invention are antibodies directed against a protein that is unaffected in the presence of acid. In fact, for the method to be specific, it is appropriate to use, as binding partner to the IgA antibody, at least the protein that is recognized by the antibody, or a fragment thereof. This protein, brought into contact with the pretreated sample, therefore at acid pH, must withstand this acidic condition.

“Protein unaffected in the presence of acid” means any protein whose three-dimensional structure undergoes little if any change at acid pH. This stability, which corresponds to resistance to acid denaturation, can be demonstrated by any known method, for example by microcalorimetry of the DSC (Differential Scanning Calorimetry) type. Briefly, this method consists of forcing the thermal denaturation of a protein inside a calorimeter in a medium having the desired pH, and deducing the two parameters, namely Tm and ΔH, from measurements that are carried out. Tm is a semi-empirical parameter, which corresponds to the temperature at which 50% of the protein is in the native state and 50% is in the denatured state. ΔH (change in enthalpy, or heat of denaturation) is a quantitative parameter corresponding to the energy that has to be supplied to the protein to obtain its complete denaturation. These two parameters are directly linked to the intrinsic stability of the protein being tested: the more their respective values increase, the greater the stability of the protein relative to a reference value.

To determine whether the three-dimensional structure of a protein is or is not affected in the presence of acid, it is necessary to determine the Tm and/or ΔH of this protein at acid pH and at its pH of immunologic stability. In this comparative experiment, the pH is the only variable factor. The concentration of the protein to be investigated, the heating rate and the chemical nature of the buffer must all be constant. Thus, buffering agents must be selected with several pKa values, giving a wide range of buffering effect, such as phosphate (1.7-2.9; 5.8-8.0), citrate (2.2-6.5) or succinate (3.2-5.2; 5.5-6.5), or use of a tri-buffer that can cover a range pH3-pH10. The acid pH to be selected for this comparative experiment is the pH of the acidic sample treatment buffer according to the invention. It is considered that the structure of the protein is not affected in the presence of acid when the ΔH determined at acid pH does not differ by at most about 25%, preferably at most about 10%, relative to the ΔH determined at the pH of immunologic stability of the protein. If it is Tm that has been calculated, the Tm determined at acid pH must not differ by at most about 25%, preferably by at most about 10%, relative to the Tm determined at the pH of immunologic stability of the protein under investigation.

Proteins that are not affected in the presence of acid are widely known by a person skilled in the art. As examples, we may mention the proteins of microorganisms, such as those of bacteria and viruses, in particular of bacterial and viral structures, apart from the envelopes, for example the Core protein of the HCV virus, as well as toxins such as toxins A and B of Clostridium difficile.

The features described above in the context of the method for predicting the susceptibility of an at-risk patient for developing or redeveloping Clostridium difficile infection apply to the method for determining the level of IgA described here, for example the immunoassay itself, the nature of the acidic sample treatment buffer, its pH, whether or not to use separation by filtration or by sedimentation of the reaction mixture after acid treatment.

In particular, as above, the duration of the pretreatment must be reasonable, notably less than 30 min, in contrast to the teachings of Tress U. et al., 2006, who recommend overnight treatment of stool samples from a dog for quantifying total IgA and a neutralizing step before immunoassay.

All the IgA antibodies of diagnostic or prognostic interest and directed against a protein that is unaffected in the presence of acid, including those directed against toxin B of Clostridium difficile, preferably secretory, are suitable for the purposes of this method. Moreover, biological samples other than stool and rectal enema, such as mucus, sputum, bronchoalveolar wash, vaginal secretions, vaginal enema, sweat, tears, saliva, milk, and colostrum, are also suitable.

Kits for determining, by immunoassay, the level of at least one IgA antibody directed against a protein that is unaffected in the presence of acid, notably IgA antibodies against toxin B of Clostridium difficile, in a patient's biological sample that may contain said at least one IgA, comprising or containing (i) one or more binding partners to said at least one IgA antibody for performing the immunoassay, and (ii) an acidic sample treatment buffer, preferably at pH 2.5, it being understood that said kit does not contain any neutralizing solution, constitute another object of the invention.

The components and characteristics of this kit are as defined above.

According to a particular embodiment, the kits also comprise or contain at least one control sample, which is a sample containing a known amount of IgA antibodies against toxin. B of Clostridium difficile.

The kits may also contain all the compounds necessary for demonstrating the reaction between the binding partner or partners and the target IgA antibodies, such as washing buffers or reagents allowing visualization of labeling or emission of a detectable signal.

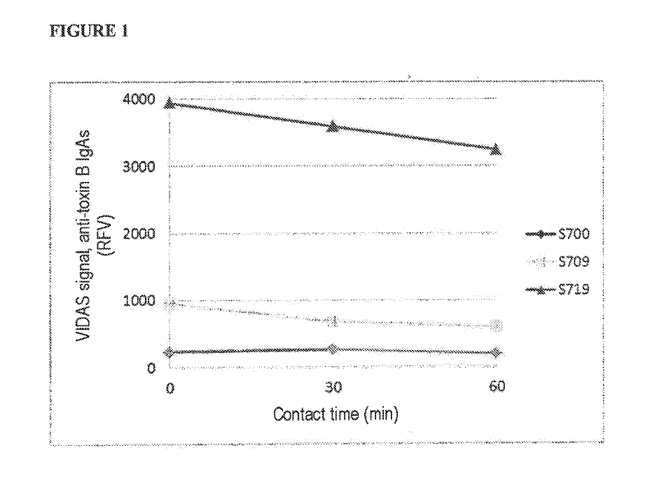

The invention will be better understood from the following examples, which are given for purposes of illustration and are nonlimiting, as well as from FIG. 1, which shows a graph giving the RFV signal obtained using the VIDAS® instrument, corresponding to the level of IgA antibodies against toxin B of 3 stool samples (S719-high positive sample; S709-low positive sample; and S700-negative sample) brought into contact with an acidic sample treatment buffer prior to the immunoassay, as a function of the duration of contact between the sample and the acidic sample treatment buffer.

### EXAMPLES

### Example 1

### Preparation of Recombinant Toxins A and B of Clostridium difficile

The tcdA gene coding for toxin A of Clostridium difficile and the tcdB gene coding for toxin B of Clostridium difficile were derived from the reference strain VPI 10463, toxinotvpe 0, ribotype 087-(toxin A: Swissprot accession No. P16154 and toxin B: Swissprot accession No. P18177). For each of the genes, the whole sequence was optimized by Geneart (Invitrogen) for expression in E. coli and 8 histidines were added on the N-term side to allow purification by metal-chelate affinity chromatography. The synthetic genes obtained were cloned into the pET3d vector (Novagen) by means of the NcoI and BamHI restriction sites.

The expression plasmids thus constructed are introduced into E. coli BL21 bacteria and derivatives (Stratagene, Agilent Technologies). The cultures are carried out in 2× YT medium (Difco), in the presence of 100 μg/mL, ampicillin and 10% glucose, at 24° C. with stirring. Induction of expression of the protein is effected by adding 1 mM of IPTG (isopropyl beta-D-1-thiogalactopyranoside) for 4 h, once it is in the exponential growth phase. At the end of culture, the bacteria are collected by centrifugation at 10000 g, at 4° C., for 30 min. The bacterial deposits are frozen at −80° C.,

The bacterial deposits are taken up in PBS buffer 2× (phosphate buffered saline) and lysed. The lysates are centrifuged at 3000 g for 30 min at 4° C. The supernatant contains the purified soluble proteins, recombinant toxin A or toxin B depending on the expression plasmid used initially.

The proteins are purified by one-step metal-chelate affinity chromatography. The supernatant obtained after centrifugation is loaded on an Ni-NTA-Agarose resin (Qiagen). After a washing cycle, the protein (toxin A or toxin B) is eluted in the presence of 250 mM imidazole. The protein is dialyzed in a 50 mM phosphate, 150 mM NaCl buffer,

### Example 2

### Immunoassay for Detecting Fecal IgA Against Toxin B

The immunoassays were carried out using the VIDAS® automatic immunoanalyzer (bioMérieux). The disposable cone serves both as solid phase for the reaction and as pipetting system. The cartridge comprises 10 wells (X0 to X9) covered with aluminum foil, sealed and labeled. The first well (X0) comprises a pre-cut part to facilitate sample insertion. The last well (X9) is an optical cuvette in which the fluorescence of the substrate is measured. The various reagents required for the analysis are contained in the intermediate wells (X1 to X8). All the steps of the assay are carried out automatically by the instrument. They consist of a succession of cycles of aspiration/discharge of the reaction mixture.

a) Sensitization and Passivation of the Cones

The cones were sensitized with 300 μL of a solution of recombinant or native toxin B diluted to 2 μg/mL in PBS buffer, pH 7.2. The inactivated native toxin B (Cat. No. CDB-TDL) was obtained from The Native Antigen Company (Upper Heyford, United Kingdom). After about 20 h of incubation at +18/25° C. with the sensitizing solution, the cones are emptied. Then 300 μL of a solution of 200 mM Tris containing 5 g/L of bovine albumin is added. Passivation is continued at +18/25° C. overnight. The cones are emptied, dried, and then stored at +4° C. until use, away from moisture.

b) Sample Pretreatment

One volume of stool is brought into contact with 3 volumes of a sample treatment buffer (dilution ¼). Improvement of the composition of this buffer is illustrated in example 3. Manual homogenization is carried out, followed by stirring in a Vortex. This step takes between about 30 seconds and 2 minutes, depending on stool consistency. The samples are centrifuged for 5 minutes at 12000 g to recover the soluble proteins. Immunoassay is carried out on this supernatant, which is deposited directly in well X1 of the VIDAS® cartridge.

c) Immunoassay Procedure

Well X1 contains 300 μL of sample diluent, with composition identical to the sample treatment buffer. 200 μL of the supernatant obtained in step b) is transferred to well X1 in order to perform additional dilution. As soon as the VIDAS® cone is in contact with the sample, the first step of the immunologic reaction begins. This step allows specific binding of fecal anti-toxin B IgAs, present or not in the stool sample supernatant, to the toxin B adsorbed on the cone. After 4 minutes of incubation at 37° C., the components that are not bound are removed by washing with 200 mM Tris buffer pH 7.8, NaCl 300 mM, Tween® 20 0.25%. In the second step, the cone is incubated with a solution of conjugate containing about 0.5 μg/mL of an anti-human IgA mouse IgG (bioMérieux) coupled to alkaline phosphatase, in a 10 mM phosphate buffer, containing 300 mM of NaCl and 5 g/L of bovine serum albumin. Well X5 contains 400 μL of this solution, which the cone aspirates/discharges for 5 minutes, still at 37° C. The second step results in the formation of a complex between the fecal anti-toxin B IgAs and the anti-IgA conjugate coupled to alkaline phosphatase. This step is followed by 3 successive washing operations to remove the compounds that are not fixed.

In the final detection step, the substrate 4-methylombelliferyl phosphate is aspirated and then discharged in the cone; the enzyme of the conjugate catalyzes the reaction of hydrolysis of this substrate to 4-methylombelliferone, whose fluorescence emitted is measured at 450 nm. The value of the fluorescence signal (RFV=relative fluorescence value) is proportional to the concentration of fecal anti-toxin B IgAs present in the sample.

Table I below presents the fluorescence signals (RFV=relative fluorescence value) determined by the VIDAS® automatic instrument using buffer R1 as sample treatment buffer. This buffer forms part of the VIDAS® C. difficile Toxin A&B kit (Cat. No. 30118, bioMérieux) for detecting toxins A and B in the stool. Its pH is 7.2, as described in the instructions with the kit. It is used for extracting the toxins from human stool. The stool samples used were obtained from patients who consulted the department of Dr. Robert Spencer, Health Protection Agency Regional Laboratory, Bristol, United Kingdom with suspected Clostridium difficile infection.

TABLE 1
Detection of the fecal IgAs in human stool.

| | Anti-toxin B IgAs | Anti-toxin B IgAs | | |
|---|---|---|---|---|
| Sample | VIDAS ® signal | Interpretation of | VIDAS ® | VIDAS ® |
| code | (RFV) | the assay | GDH | Tox A & B |
| S700 | 36 | Neg | Neg | ND |
| S706 | 9 | Neg | Neg | ND |
| S709 | 114 | Pos | Neg | ND |
| S713 | 91 | Neg | Pos | Neg |
| S719 | 831 | Pos | Neg | ND |
| S720 | 56 | Neg | Pos | Neg |

Neg = negative;
Pos = positive;
ND = not determined

The result of the immunoassay is interpreted by comparing the RFV signal measured for each sample at the limit of detection of the VIDAS® assay, which is defined as follows; mean background noise (RFV signal measured with a buffer) +3 standard deviations. The VIDAS® signals above this limit of detection are regarded as positive: anti-toxin B IgA antibodies are present. The VIDAS® signals below this limit of detection are regarded as negative: no anti-toxin B IgA antibodies are present.

It is important to note that sample S709 with a signal of 114 RFV is classified as positive whereas sample S713 with a signal of 91 RFV is classified as negative. Two signals that are close in absolute value lead to a different biological interpretation: this situation is not satisfactory. It therefore needs to be improved.

### Example 3

### Optimization of Extraction of the Fecal IgAs

Buffer R1 suitable for extraction of toxins A and B, used in example 2, showed that it was possible to improve the analysis of the fecal IgAs. Another sample treatment buffer was therefore tested using the stool samples characterized in example 2, with the same treatment time as before (30 s and 2 min). This buffer is a hydrochloric acid-glycine buffer at pH 2.5 (1M glycine pH 2.5). Table 2 below presents the fluorescence signals (RFV=relative fluorescence value) determined by the VIDAS® automatic instrument. The results presented in this experiment were obtained using cones on which recombinant toxin B was immobilized.

TABLE 2
Detection of the fecal IgAs extracted with different
buffers from human stool. Acid buffer = 1M glycine
pH 2.5 and buffer R1 described in example 2.

Anti-toxin B IgAs,
VIDAS ® signal (RFV)

| Sample | Prescence | Buffer R1 | |
|---|---|---|---|
| code | of IgA | (state of the art) | Acid buffer |
| S700 | Neg | 36 | 127 |
| S706 | Neg | 9 | 60 |
| S713 | Neg | 91 | 137 |
| S720 | Neg | 56 | 169 |
| S709 | Pos | 114 | 1303 |
| S719 | Pos | 831 | 2797 |

Pos = positive;
Neg = negative

The 1M glycine acidic buffer pH 2.5 makes it possible to increase the RFV signals obtained for the positive samples significantly, from 3 to 7 times more than with buffer R1. The extraction solution at acid pH is selected for the rest of the experiments.

### Example 4

### Investigation of the Contact Time Between the Sample and the Sample Treatment Buffer

The duration of contact between the sample and the acidic sample treatment buffer (condition in example 3) was studied using a high positive sample (S719), a low positive sample (S709) and a negative sample (S700). The results are presented in FIG. 1. The negative sample (S700) did not present a significant decrease in signal. The positive samples showed a decrease of the signal. After 30 minutes of incubation, there is still 91% of the signal for S719 and there is still 72% of the signal for S709. After 60 minutes, there is still 82% of the signal for S719 and 63% of the signal for S709. These results show that incubation for 60 min in the presence of the 1M glycine buffer pH 2.5 causes a significant decrease in the amount of anti-toxin B IgAs present in the sample. In the case of weakly positive samples, it would be best not to exceed 15 min so as not to lose more than 10% of the signal. Such a sample could be detected as “negative” if the duration of contact with the extraction buffer is prolonged excessively. For the rest of the experiments, we used a contact time from about 30 seconds to 2 minutes.

### Example 5

### Comparison of Recombinant Toxin B and Native Toxin B for Detecting the Anti-Toxin B IgAs by Immunoassay

We compared the fluorescence signals obtained when using a recombinant toxin B and a native toxin B for preparing the capture phase (adsorption on the cones). The two types of toxin B were immobilized on the solid phase at the same concentration (2 μg/mL). The results are presented in Table 3 below. Samples SP023 and SP pool were obtained from Professor M. Delmée, Clostridium difficile National Reference Center, Catholic University of Louvain, Brussels, Belgium.

The procedure described in example 3 above was repeated.

Apart from sample S709, the VIDAS® signals obtained for all the samples are stronger when the solid phase has the native toxin than the recombinant toxin. For the negative samples, the gain is negligible (+37 to +67 RFV), whereas for 3 of the 4 positive samples the gain is very large (+640 to +1797 RFV). Thus, use of the native toxin B for capture allows better discrimination of the positive and negative samples.

According to the results presented in Table 3, the positivity threshold when using recombinant toxin B is fixed at 200 RFV whereas the positivity threshold when using native toxin B is fixed at 250 RFV.

TABLE 3
Detection of the fecal anti-toxin B IgAs by recombinant
toxin B or native toxin B. Pretreatment of the stool
samples was carried out in the presence of 1M glycine
acidic buffer pH 2.5, according to example 3.

| | | Anti-toxin B IgAs, | | |
|---|---|---|---|---|
| | | VIDAS ® signal (RFV) | | |
| Sample | Presence | Recombinant | Native | ΔSignal |
| code | of IgA | Toxin B | Toxin B | Native − Recomb. |
| S700 | Neg | 127 | 194 | +16 |
| S706 | Neg | 60 | 97 | +37 |
| S713 | Neg | 137 | 193 | +56 |
| S720 | Neg | 169 | 215 | +46 |
| S709 | Pos | 1303 | 623 | −680 |
| S719 | Pos | 2797 | 3437 | +640 |
| SP023 | Pos | 498 | 1221 | +723 |
| SP pool | Pos | 1637 | 3434 | +1797 |

Pos = positive;
Neg = negative

### Example 6

### Investigation for Fecal IgAs in Patients with Multiple Reinfection and Determination of the Reference Values S

The stool samples used in this example were obtained from patients who had been infected repeatedly with Clostridium difficile, called patients with multiple reinfection. These patients consulted the Clinical Microbiology Division, in the Medical Department of the Sir Mortimer B. Davis—Jewish General Hospital, in Montreal, Quebec, Canada. Pretreatment of the stool samples was carried out in the presence of the 1M glycine acidic buffer pH 2.5, according to the conditions in example 3. The different types of toxin were all immobilized on the solid phase at the same concentration (2 μg/mL). The fecal IgAs against toxin B, as well as the fecal IgAs against toxin A, were investigated by immunoassay using the toxins described in example 1 according to the procedure described in example 2. The results are presented in Table 4.

TABLE 4
Investigation for fecal IgAs against toxin B and against
toxin A in the patients' stools with multiple reinfection.

| | VIDAS ® signal (RFV) | | VIDAS ® signal |
|---|---|---|---|
| | Anti-toxin B IgAs | | Anti-toxin A IgAs |
| | Native | Recombinant | Recombinant |
| Sample code | Toxin B | Toxin B | Toxin A |
| MR001 | 11 | 5 | 6 |
| MR002 | 867 | 191 | 156 |
| MR003 | 97 | 35 | 66 |
| MR004 | 129 | 37 | 60 |
| MR005 | 161 | 42 | 60 |
| MR006 | 20 | 14 | NAv |
| MR007 | 380 | 145 | 138 |
| MR008 | 337 | 214 | NAv |
| MR009* | 1001 | 609 | 462 |

NAv = not available, the test could not be carried out, owing to insufficient sample volume.

In 3 patients with multiple reinfection, among the 8 tested, it was possible to detect fecal IgAs against toxin B (MR002, MR007, MR008). For all these patients, the signal obtained is above the positivity threshold of the assay, of 250 RFV. Moreover, these results confirm independently that the immunoassay using recombinant toxin B is less sensitive than the method using native toxin B: patient MR007 is not detected as positive and patient MR002 is at the limit of positivity with a signal of 191 RFV, for a threshold at 200 RFV.

Clinical follow-up of the patients showed that all the patients MR001 to MR008 presented at least one reinfection after their consultation and obtaining of the samples used in this example. Our assays show that some of these patients (MR002, MR007, MR008) have anti-toxin B IgA antibodies, and in some cases anti-toxin A IgA antibodies, or neither of these two types of antibodies. It may be concluded that the titer of the fecal anti-toxin B IgA antibodies that these patients possess is not sufficient to protect against such a reinfection. This group of patients therefore constitutes the group of patients with increased risk of developing or redeveloping Clostridium difficile infection.

Patient MR009 was initially classified in the group of patients with multiple reinfection according to the clinical picture. Supplementary biological investigations showed that this is in fact a poor classification. This patient was monitored for 3 months after this first assay. Investigation for the bacterium Clostridium difficile by culture always proved negative despite several attempts. No reinfection was observed. It should therefore be considered that patient MR009 is protected against reinfection. This patient has both anti-toxin B IgAs (1001 RFV) and anti-toxin A IgAs (462 RFV). This patient therefore constitutes the group of patients without an increased risk of developing or redeveloping Clostridium difficile infection.

Based on all of these results obtained for these groups of patients, we are able to define the reference value S. For a given capture antigen, the reference value S is selected in such a way that all the VIDAS® signals obtained for the patients with multiple reinfection MR001 to MR008 are below it and those for patient MR009 are above it.

For the anti-toxin B antibodies, the reference value S is between 870 and 1000 RFV, or fixed at about 930 RFV, when a native toxin B is used for capture. It is between 200 and 600 RFV, or fixed at about 400 RFV, when recombinant toxin B is used for capture. For the anti-toxin A antibodies, the reference value S is between 160 and 460 RFV, or fixed at about 310 RFV, when a recombinant toxin A is used for capture.

### Example 7

### Investigation for Fecal IgAs in Toxin A and B Negative Patients with Suspected Clostridium difficile Infection

The stool samples used in this example were obtained from patients who presented with suspected Clostridium difficile infection, in the department of Dr. Robert Spencer, Health Protection Agency Regional Laboratory, Bristol, United Kingdom. The samples in our possession were characterized using the kits VIDAS® C. difficile GDH (Cat. No. 30125, bioMérieux) and VIDAS® C. difficile Toxins A and B (Cat. No. 30118, bioMérieux). Investigation for fecal IgAs was carried out according to the protocols described in examples 2, 3 and 6 using native toxin B and recombinant toxin A.

The samples selected for the investigation in example 7 are all toxin A and toxin B negative, as well as GDH negative (Table 5). This selection includes two subgroups of patients. In a first subgroup, these two assays are negative because the patients had never been in contact with the bacterium C. difficile. The symptoms that led to suspicion of infection with C. difficile are due to another cause. The second subgroup corresponds to the patients who had actually been in contact with the bacterium but were able to eliminate it or limit its multiplication and thus recover from infection. We do not have the clinical data that would allow us to distinguish between these two subgroups. In contrast, if we detect anti-toxin B IgAs and/or anti-toxin A IgAs in the patients' stool, they are undoubtedly patients who have had a symptomatic infection or are carriers of Clostridium difficile. Thus, we showed that 6 patients out of the 12 tested (50%) have levels of anti-toxin B antibodies above the reference value S defined in example 6. Of these 6 patients, only 5 also have anti-toxin A antibodies that are present above the reference value S defined in example 6 (Table 5). Thus, if only investigated for anti-toxin A IgAs, patient SP252 would not be detected as having anti-Clostridium difficile secretory antibodies.

The results obtained for this cohort show that detection of the anti-toxin B IgAs is more sensitive than detection of the anti-toxin A IgAs.

TABLE 5
Investigation for fecal IgAs against toxin B and toxin A in the stool
from patients with suspected Clostridium difficile infection.

| | | | | | Interpretation (relative to the | |
|---|---|---|---|---|---|---|
| | | | VIDAS ® signal (RFV) | | reference value) | |
| Sample | VIDAS ® | VIDAS ® | Anti-toxin | Anti-toxin | Anti-toxin | Anti-toxin |
| code | GDH | CDAB | B IgAs | A IgAs | B IgAs | A IgAs |
| SP29 | Neg | Neg | 2034 | 390 | Pos | Pos |
| SP32 | Neg | Neg | 2094 | 1056 | Pos | Pos |
| SP37 | Neg | Neg | 84 | 24 | Neg | Neg |
| SP38 | Neg | Neg | 108 | 60 | Neg | Neg |
| SP40 | Neg | Neg | 9684 | 4338 | Pos | Pos |
| SP43 | Neg | Neg | 1044 | 378 | Pos | Pos |
| SP228 | Neg | Neg | 24 | 18 | Neg | Neg |
| SP232 | Neg | Neg | 12 | 18 | Neg | Neg |
| SP234 | Neg | Neg | 42 | 24 | Neg | Neg |
| SP236 | Neg | Neg | 2274 | 636 | Pos | Pos |
| SP251 | Neg | Neg | 36 | 24 | Neg | Neg |
| SP252 | Neg | Neg | 1026 | 240 | Pos | Neg |

Pos = positive;
Neg = negative

### Example 8

### Investigation for Fecal IgAs in Patients with Clostridium Difficile Infection and Prediction of Reinfection

The stool samples used in this example were obtained from patients with proven Clostridium difficile infection. These patients presented in the Clinical Microbiology Division, in the Medical Department of the Sir Mortimer B. Davis—Jewish General Hospital, Montreal, Quebec, Canada. Wherever possible, two consecutive stool samples, from 10 to 35 days apart, were collected for each patient. The number of days between the first sample and the second is shown in Table 6. For all the patients, PCR investigation for the genetic material of the bacterium Clostridium difficile was carried out on the extracts from the first stool samples. This investigation proved positive for all the patients included in the study presented here, thus proving the clinical suspicion of Clostridium difficile infection.

The investigation for fecal IgAs was carried out according to the protocols described in examples 2, 3 and 6 using native toxin B. The samples for which we obtain a signal above the reference value S determined in example 6 (930 RFV when native toxin B is used for capture) are regarded as positive and those for which the signal is below this value are negative. The results are presented in Table 6.

TABLE 6
Investigation for fecal IgAs against toxin B in the stool of patients with Clostridium difficile infection.

| | 1st sample | | 2nd sample | | | | | Clinical |
|---|---|---|---|---|---|---|---|---|
| | Anti-toxin B IgAs | | | | Anti-toxin B IgAs | | Risk of | Number of |
| Patient | RFV | | | VIDAS ® | RFV | | reinfection | reinfections |
| code | signal | Interpretation | Day | CDAB | signal | Interpretation | (prediction) | observed |
| CD001 | 10451 | Pos | NA | NAv | NAv | NAv | NAv | No follow-up |
| CD002 | 3623 | Pos | NA | NAv | NAv | NAv | NAv | 1 |
| CD003 | 413 | Neg | +13 | NAv | 2708 | Pos | Low | 1 |
| CD004 | 675 | Neg | +12 | Pos | 3291 | Pos | Low | 1 |
| CD005 | 1891 | Pos | NA | NAv | NAv | NAv | NAv | No follow-up |
| CD006 | 6881 | Pos | +19 | Pos | 2254 | Pos | Low | 1 |
| CD007 | NAv | NAv | +34 | Pos | 7528 | Pos | Low | 1 |
| CD008 | 3480 | Pos | +25 | NAv | 922 | Neg | High | 2 |
| CD009 | 2107 | Pos | +17 | Pos | 1475 | Pos | Low | 1 |

Pos = positive;
Neg = negative;
NAv = not available, the test could not be carried out, owing to insufficient sample volume;
NA = not applicable

To evaluate the risk of reinfection, it is useful to investigate for fecal IgAs against toxin B in a stool sample obtained 10 to 38 days, preferably 15 to 25 days after the infection with Clostridium difficile regarded as primary infection.

If the stool sample contains a level of anti-toxin B IgAs above the reference value S, the patient is at LOW risk of reinfection: he will have 0 or 1 reinfection at most after the episode of infection with Clostridium difficile regarded as primary infection.

If the stool sample contains a level of anti-toxin B IgAs below the reference value S, the patient is at HIGH risk of reinfection: he will have 2 reinfections or more after the episode of infection with Clostridium difficile regarded as primary infection.

The predictions made by applying this rule are presented in the column “Risk of reinfection” in Table 6, which also shows the number of reinfections actually observed in these same patients. It will be noted that there is perfect agreement between the predictions and the clinical observations.

For 3 patients (CD001, CD002 and CD005), it was not possible to obtain a second sample 10 to 35 days after the first. However, the analyses show that the first stool samples obtained from these patients contain high levels of fecal anti-toxin B IgAs.

### REFERENCES

Bartlett, J. G., 2002, N. Engl. J. Med. 346 (5): 334-339
Bauer M. P. et al., 2011, Lancet, 14: 63-73
Eyre D. W, et al., 2012, Clinical Infectious Diseases, 55(S2): 77-87
Freeman J., 2010, Clin. Microbiol, 3: 529-549
Garey K. W. et al., 2008, Journal of Hospital Infection, 70: 142-1.47
Johnson S. et al., 1992, JID, 166: 1287-1294
Kelly C. P. and Lamont. J. T., 2008, N Engl. J. Med., 359: 1932-1940
Kuijper E. J. et al., 2006, Clinical Microbiology and Infectious Diseases, CMI, 12(S6): 2-18
Tress U, et al., 2006, AJVR, 67(10): 1756-1759
Warny M. et al., 1994, Infection and Immunity, 62(2): 384-389

1. A method for predicting the susceptibility of an at-risk patient for developing or redeveloping Clostridium difficile infection, comprising determining, by immunoassay, in a stool sample from said patient, the level of IgA antibodies against toxin B of Clostridium difficile, and comparing this level against a reference value S determined beforehand with two populations of patients exposed to the bacterium, one not having developed or redeveloped such an infection and the other having developed or redeveloped such an infection,
a level below said reference value S signifying that the patient is a patient with increased risk of developing or redeveloping Clostridium difficile infection, and
a level above said reference value S signifying that the patient is not a patient with increased risk of developing or redeveloping Clostridium difficile infection.

2. The method as claimed in claim 1, wherein the stool sample used in the immunoassay had been treated beforehand with an acidic sample treatment buffer, preferably at pH 2.5.

3. The method as claimed in claim 2, wherein, in the sample pretreatment step, the sample is brought into contact with the acidic sample pretreatment buffer for at most 30 min, and it then undergoes separation, notably by filtration or sedimentation, the filtrate or supernatant then being recovered, to be used in the immunoassay.

4. The method as claimed in claim 2, wherein the sample pretreatment step does not employ a neutralizing step before the pretreated sample is used in the immunoassay.

5. A method for determining, by immunoassay, the level of at least one IgA antibody directed against a protein that is unaffected in the presence of acid, preferably directed against toxin B of Clostridium difficile, in a patient's biological sample that may contain said at least one IgA antibody, comprising bringing one or more binding partners to said at least one IgA antibody, used for performing the immunoassay, into contact with an acidic reaction mixture comprising said biological sample pretreated with an acidic sample treatment buffer, without neutralization before it is used in the immunoassay.

6. The method as claimed in claim 5, wherein, the sample pretreated with the acidic buffer undergoes separation, notably by filtration or sedimentation, the filtrate or supernatant then being the reaction mixture recovered for use in the immunoassay.

7. The method as claimed in claim 5, wherein the acidic sample pretreatment buffer has pH 2.5.

8. A kit for determining, by immunoassay, the level of at least one IgA antibody directed against a protein that is unaffected in the presence of acid, notably directed against toxin B of Clostridium difficile, in a patient's biological sample that may contain said at least one IgA antibody, comprising
(i) one or more binding partners to said at least one IgA antibody for performing the immunoassay, and
(ii) an acidic sample treatment buffer, it being understood that said kit does not contain any neutralizing solution.

9. The kit as claimed in claim 8, in which the biological sample consists of stool or rectal enema from the patient.

10. The kit as claimed in claim 8, for immunoassay detection of IgA antibodies against toxin B of Clostridium difficile in a patient's stool sample that may contain said IgAs, also comprising
(iii) at least one control sample, which is a sample containing a known amount of IgA antibodies against toxin B of Clostridium difficile.
